(19) United States
(12) Patent Application Publication
(10) Pub. No.: US 2025/0152105 A1
(43) Pub. Date: May 15, 2025

(54) EARLY CRITICAL EVENT DETECTION AND MITIGATION SYSTEM FOR PATIENTS IN INTENSIVE CARE UNITS

(71) Applicant: SPASSMED INC., Vaughan (CA)

(72) Inventors: Anubhav BHATTI, Toronto (CA); Surajsinh PARMAR, Toronto (CA); Divij GUPTA, Toronto (CA); Jong KO, Toronto (CA)

(73) Assignee: SPASSMED INC., Vaughan (CA)

(21) Appl. No.: 18/942,328

(22) Filed: Nov. 8, 2024

Related U.S. Application Data
(60) Provisional application No. 63/597,389, filed on Nov. 9, 2023.
(60) Provisional application No. 63/615,405, filed on Dec. 28, 2023.
(60) Provisional application No. 63/660,399, filed on Jun. 14, 2024.

(51) Int. Cl. A61B 5/00; G06N 3/09; G16H 15/00; G16H 50/50

(52) U.S. Cl. CPC A61B 5/7275; G06N 3/09; G16H 15/00; G16H 50/50

(57) ABSTRACT

Provided are a method, device, and recording medium of early prediction of sepsis or septic shock through bio-data analysis on a computing device. In an embodiment, the method comprises training one or more time series deep learning models by: encoding sepsis or septic shock patient vital signs as time series-based bio-data to generate embedded time series patches; encoding associated patient medical histories utilizing a pre-trained language encoder to generate encoded patient medical history patches; combining the embedded time series patches and the patient medical history patches into an array; and randomly masking the embedded time series patches and asking the model to reconstructing the masked patches based at least in part on the associated patient medical histories; and utilizing the one or more trained time series deep learning models to predict sepsis or septic shock for a new patient.

### CROSS-REFERENCE TO RELATED APPLICATIONS

This application claims the benefit of U.S. Prov. Appl. No. 63/597,389 filed on 2023 Nov. 9 and entitled Early Critical Event Detection and Mitigation System for Patients in Intensive Care Units; U.S. Prov. Appl. No. 63/615,405 filed on 2023 Dec. 28 and entitled Advanced Critical Event Detection and Mitigation System for Patients In Intensive Care Units; and U.S. Prov. Appl. No. 63/660,399 filed on 2024 Jun. 14 and entitled Early Critical Event Detection and Mitigation System for Patients In Intensive Care Units, the entirely of which are incorporated herein by reference.

### FIELD

This application relates to the field of artificial intelligence (AI) systems and methods for healthcare, and more particularly to a system and method for early detection of critical events.

### BACKGROUND

Sepsis and septic shock are life-threatening conditions that result in a high mortality rate in hospitals. Time-sensitive intervention is crucial for improving patient outcomes, but conventional methods for detecting sepsis may be delayed by unclear symptoms, leading to a significant number of deaths. Laboratory tests and scoring systems like qSOFA and SIRS are not consistently effective in detecting sepsis, and the mortality rate for these conditions remains high, accounting for between 24.4% and 38.8% of patient deaths, according to Critical Care. Therefore, there is a pressing need for more effective and efficient methods of detecting sepsis and septic shock to reduce mortality rates and improve patient outcomes.

### SUMMARY

This disclosure relates to an advanced AI healthcare monitoring framework designed to detect and mitigate the onset of critical diseases and events, such as sepsis and septic shock in patients, by providing vital clinical decision support to healthcare professionals. It harnesses a suite of deep learning (DL) models that process a range of patient data, including vital signs, demographics, medical history, and social habits, to predict and alert healthcare workers of potential acute events hours in advance. The system includes a DL classifier to identify disease onset, a forecaster to project vital signs for the subsequent hours, an anomaly detector that evaluates both real-time and forecasted vital signs to estimate the likelihood of critical events, and a generative AI model that synthesizes all this data into comprehensive reports, providing diagnostic and treatment plans with reference sources. This integrated approach aims to mitigate risks and improve patient outcomes by enabling early and informed intervention.

In an embodiment, Parameter-Efficient Fine-Tuning (PEFT) techniques are used to selectively fine-tune a subset of weights within a pre-trained large time-series encoder on domain-specific data. Following this approach, a minimal subset of weights, pre-existing within the encoder architecture or newly introduced, is fine-tuned, while the remainder of the model weights are frozen. This approach leverages robust feature representations acquired during initial pre-training while allowing for the incorporation of domain-specific adaptations through selective fine-tuning. As a result, the computational requirements and inefficiencies associated with fine-tuning the entire encoder on a specific task or dataset are significantly reduced.

### BRIEF DESCRIPTION OF THE DRAWINGS

FIG. 1 shows an artificial intelligence (AI) pipeline for a novel system for early detection and mitigation of the onset of diseases in accordance with an embodiment.

FIG. 2 shows a prototype GUI of the overall AI early warning dashboard.

FIG. 3 shows a schematic diagram of a pipeline for encoding a patient's vital signs and clinical history, process it, and subsequently forecast the vital signs, detect onset of sepsis, predict occurrence of septic shock, and generate the patient report.

FIG. 4 shows a block diagram showing a position-wise feed-forward (PFF) layer in accordance with an illustrative embodiment.

FIGS. 5A-5D and FIG. 6 show schematic diagrams of another illustrative embodiment.

FIG. 7 shows a generic computing device which may provide a suitable operating environment for one or more embodiments of the invention.

### DETAILED DESCRIPTION

The present application discloses a method and system for early detection and mitigation framework for the onset of diseases, e.g., sepsis and septic shock in patients. The method and system provides clinical decision support to the healthcare workers if an acute event is predicted, e.g., generating diagnosis report and treatment plan for mitigating septic shock event in advance.

This method and system monitors the patient's information, such as vital signs, drug infusion scheme, patient demographics, medical history, social habits, etc., to predict critical events several hours (e.g., 3 hours or more) in advance. The monitored vital signs of the patients include one or more of blood pressure, systolic blood pressure, diastolic blood pressure, heart rate, respiration rate, body temperature, etc. This method or system includes one or more DL-based models, e.g., classifier, forecaster, anomaly detector, and a generative AI model, e.g., a large language model (LLM).

Illustrative embodiments will now be described with reference to the drawings.

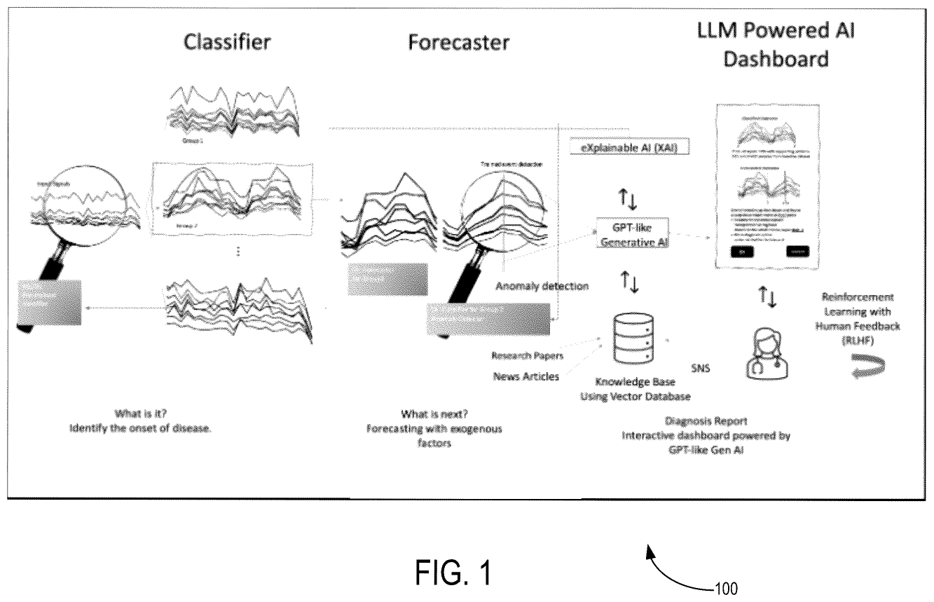

Now referring to FIG. 1, shown is an AI pipeline 100 for the novel system for early detection and mitigation of the onset of diseases. The pipeline takes the patient's information, such as vital signs, medical history, patient habits, etc., as input, and the vital sign classifier detects the onset of a critical disease. The vital sign forecaster then forecasts the vital signs for subsequent hours, and an anomaly detector detects the probability of a critical event. Based on the models' outputs and the patient's medical history, the Generative AI model generates a diagnosis report and an action plan to mitigate the critical event for healthcare professionals. The healthcare professional can record their feedback on the generated response, and a reinforcement learning (RL) agent will use this human feedback to improve the future response of the Generative AI model. This Gen AI transformer comprises a multi-modal large transformer model.

Still referring to FIG. 1, this illustrative system monitors the patient's information, such as vital signs, drug infusion scheme, patient demographics, medical history, social habits, etc., to predict critical events several hours (e.g., 3 hours or more) in advance. The monitored vital signs of the patients includes one or more of blood pressure, systolic blood pressure, diastolic blood pressure, heart rate, respiration rate, body temperature, etc. The integrated array of deep learning models may include one or more of a Deep Learning Classifier, a Deep Learning Forecaster, an Anomaly Detection Model, and a Generative AI Model. The details of these models and their functionality are provided below:

Deep Learning Classifier: The DL classifier model monitors the real-time vital signs, patient demographics, drug infusion, medical history, social habits, etc., of a patient in critical care to detect the onset of an acute disease such as sepsis. If sepsis is detected, it generates an output showing the probability of the onset of sepsis in the patient.

Deep Learning Forecaster: As soon as the deep learning classifier detects an onset of critical disease, the deep learning-based forecaster starts forecasting all the relevant vital signs of the patient. The deep learning forecaster uses the real-time collected vital signs (e.g., 3 to 6 hours of vital signs) and known patient history to perform forecast vital signs, e.g., up to 3 or 6 hours of vital signs.

Anomaly Detection Model (Septic Shock Model): As soon as the forecaster forecasts the vital signs of the patient, an anomaly detection model scans the vital signs (real-time collected vital signs and forecasted vital signs) and determines anomalies related to the acute event, e.g., septic shock and generates a probability of the critical event. If the likelihood of a critical event surpasses a certain threshold, a warning is generated.

Generative AI model: Along with the warning for a critical event, a snapshot of all the information, e.g., vital signs, patient history, drug infusion, medication history, and outputs of classifier, forecaster, and anomaly detector, is transformed into a ‘robot-language’ and fed to a Generative AI model (e.g., an LLM). The Generative AI model takes in all the transformed snapshots of the patient information and generates a diagnosis report for the healthcare workers that includes the suggested action plan, treatment plan, and assessment of the patient's condition in future. The generative AI model also generates sources for all the studies used by the model to generate recommendations for treatment and action plans. This diagnosis report aims to provide clinical decision support to healthcare workers.

The system or the method allows the healthcare professionals to provide feedback on the generated response from the Generative AI model, e.g., good or not good, or highlight issues in the generated response. The feedback received from the healthcare professionals is collected and stored in a backend database. A Reinforcement Learning loop with Human Feedback (RLHF) agent in the backend uses the feedback to fine-tune the model in a reinforced manner to further improve the response of the Generative AI model in future.

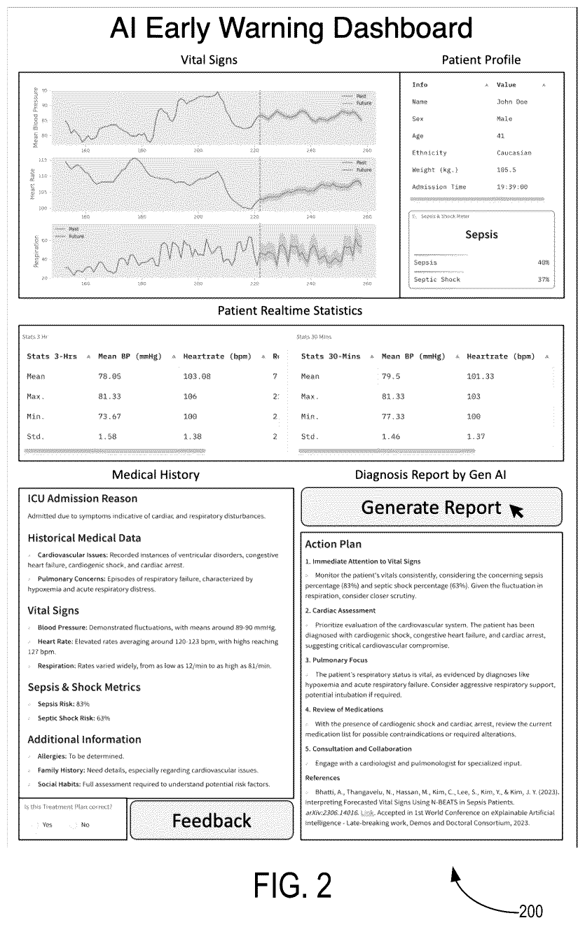

Now referring to FIG. 2, shown is an illustrative GUI 200 of the overall artificial intelligence (AI) early warning dashboard in accordance with an embodiment. Here, the monitored vital signs of the patient are shown on the top left. The patient profile comprising patient demographic information and characteristics is on the top right. Below the patient profile is the likelihood of critical events, such as sepsis and septic shock, predicted by the deep learning models. In the middle, statistical information on the vital signs is captured (e.g., last 3 hours or 30 minutes). Finally, patient history is shown on the bottom left, and the Action plan generated by the Generative AI model is offered on the bottom right. A feedback option is provided above the output of generative AI for healthcare to give feedback on the generated response of the generative AI. This feedback can be used to improve the future responses of the model.

While the identity of a patient must of course be maintained for a specific patient for whom the model is making a prediction, for data preparation purposes for training, it should be anonymized. In this illustrative example, starting from the left, vital signs data is collected from a patient, then undergoes a data masking process. Data cleaning and masking can provide various benefits, including concealing sensitive information, such as personal identifiers, financial details, or health records, to protect individual privacy. This is especially relevant when working with datasets that include personal data subject to privacy regulations (e.g., GDPR in Europe, HIPAA in the U.S., or PIPEDA in Canada). Another advantage is enhanced security. By transforming sensitive data into a format that cannot be easily reversed or decoded, data masking reduces the risk of data breaches and the potential misuse of data.

Effective data cleaning and masking techniques can also maintain the structural integrity of the data, allowing for the development and training of ML models without compromising on the accuracy or effectiveness of the models. For example, while names and specific birth dates could be stripped from the data, all other relevant information need to provide context for the vital signs data, including age, sex, BMI, and other relevant physiological information may be maintained.

The masked patient vital signs data is encoded using an encoder in order to transform the data into a format that can be understood and processed by the machine learning algorithms. This conversion of patient vital signs data into a numeric format enables machine learning models to process and interpret the patient vital signs data using these algorithms.

The choice of an encoder may depend on the nature of the vital signs (e.g., continuous vs. categorical data) and the specific requirements of the machine learning task at hand (e.g., prediction, classification, anomaly detection).

Encoding methods may include Min-Max scaling, where the minimum and maximum values are determined using clinically sensible scale ranges for each vital sign, e.g., HR from 0 to 300 bpm, mean blood pressure from 0 to 190 mmHg, etc.

Encoding methods may also include lagged features, i.e., creating another feature for each timestamp to capture the trends in the data over time. Additionally, statistical features such as mean, standard deviation, max, min, kurtosis, median, and skew were calculated for each variable using a variable running period for each hour at different periods and their crossovers.

Encoding methods may also include computing the Fourier transform of input vital signs to encode information in the frequency domain.

By way of example and not by way of limitation, encoding methods may include a Min-Max Scaler for normalizing continuous vital signs (e.g., heart rate, blood pressure, or temperature) to a specific range, say between 0 and 1. This normalization can be crucial for models sensitive to the scale of input features, such as neural networks.

As another example, a Standard Scaler (Z-Score Normalization) may be used to transform continuous data to have a mean of 0 and a standard deviation of 1. This is useful when the data across different vital signs have different scales and when the algorithm assumes the data to be normally distributed. This may facilitate faster convergence in gradient descent for models like linear regression, logistic regression, and neural networks.

Another example is One-Hot Encoding which may be suitable for categorical vital signs or statuses (like “normal,” “elevated,” “critical”) to create a binary column for each category.

As another example, Ordinal Encoding may apply to ordinal vital signs or indicators that have a clear ordering or hierarchy (e.g., pain levels from low to high). This encoding assigns a unique integer to each category based on its order. This preserves the ordinal nature of the data, which can be crucial for models to understand and leverage the inherent order in the feature.

As another example, vital sign input data, e.g., patient demographics and medical history data, can be encoded using a pre-trained large language encoder-only model. Where pre-trained large language model may be trained on Medical text corpus such as clinical notes from nurses, PubMed articles, etc.

Various encoding techniques can be used to prepare vital signs data vectors for training purposes. Representation learning is an important technique that involves mapping data, in this case, various patient vital signs data, to vectors of real numbers. These representations can help in reducing the dimensionality of the input data, converting sparse, high-dimensional data into a more compact, dense, and continuous vector space. This may make the data more manageable and computationally efficient for training. The representation mapping is learned in a pre-training routine using self-supervised learning (SSL).

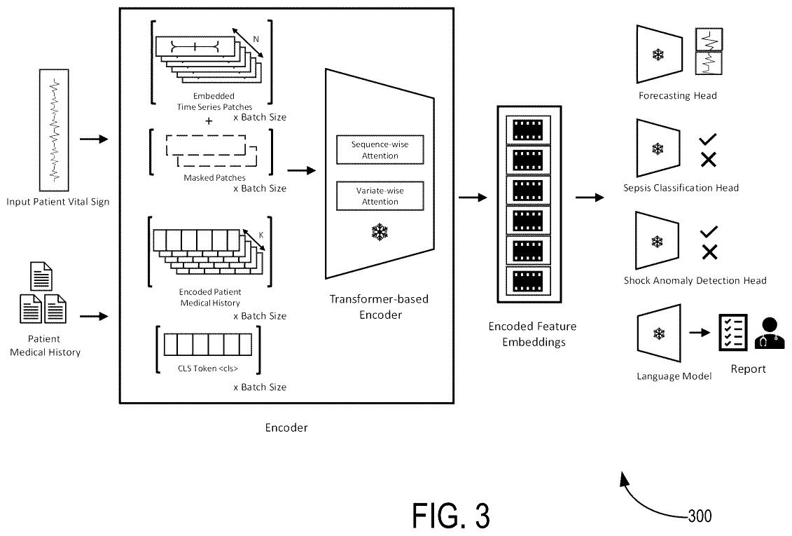

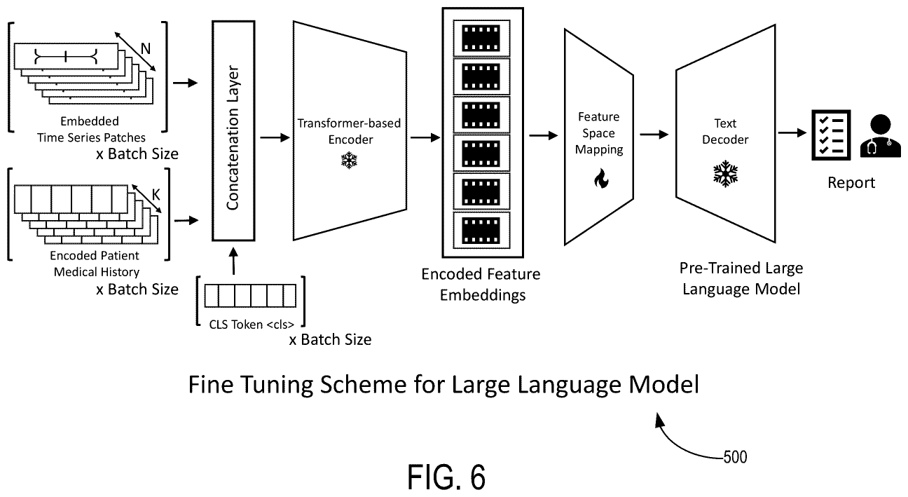

FIG. 3 describes an embodiment comprising a pipeline 300 for encoding a patient's vital signs and clinical history and subsequently forecasting the vital signs, detecting the onset of sepsis, predicting the occurrence of septic shock, and generating the patient report. This pipeline comprises three different routines: pre-training routine, supervised training routine, and inference. In the pre-training task, the encoder is trained on a large corpus of unlabeled data using SSL, where specific segments, known as patches, of the inputs (such as vital sign data, patient's other information, etc.) are randomly masked using placeholder tokens called mask tokens, and the encoder is tasked with reconstructing these segments. The encoder in this pre-training task comprises multiple sequentially connected transformer blocks that process and learn the deep representations of the input data by leveraging self-attention. The different elements of the encoder are patching layer, positional embedding layer, transformer-based encoder, and final linear layer. The input vital sign data can be represented as t can be represented as t={t1, t2, t3, . . . tT} where T is the number of timestamps in t. Instead of individually treating the vital sign time series at each timestamp, patches are created from the vital sign and feed the patches are fed to the subsequent layers for processing. This helps the model use contextual information from the patches to better learn the complete vital sign data embeddings instead of using each data point individually, which might not contain any semantic information about the sequence. Positional embeddings are added to the projections of the patch embeddings to add position/sequence ordering information to the patch embeddings, and specific learnable classification token “<cls>” (learned during training) are concatenated with the patch embeddings. Then, the embeddings are fed to a transformer-based encoder that comprises multiple transformer blocks connected in series. Each transformer block comprises various Multi-Head Self-Attention (MHSA) units, feedforward (FF) layers, normalization layers and residual connections. Where MHSA units can be applied sequence-wise, variate-wise, or as a combination of both. In each head of the MHSA unit, the Q, K, and V attention mechanisms are used, wherein query Q, key K, and value V are produced using three distinct linear transformations (learned during training). These transformations are mentioned as Q=XW_{Q}+b_{Q}, K=XW_{K}+b_{K}, and V=XW_{V}+b_{V}, where X is the matrix of input patch embeddings, and W_{Q}, W_{K}, W_{V}, are the attention weight matrices for query (Q), key (K), and value (V), respectively. b_{Q}, b_{K}, and b_{V} are optional bias terms. Further, the attention is applied as

$$Attention ( Q , K , V ) = softmax ( {QK}^{T} / √ {d}_{K} ) * V ,$$

where K^{T} implies the transpose of the key matrix, and d_{K} is the dimension of the key matrix, used as a scaling factor [see, for example, Ashish Vaswani, Noam Shazeer, Niki Parmar, Jakob Uszkoreit, Llion Jones, Aidan N. Gomez, Lukasz Kaiser, Illia Polosukhin, “Attention Is All You Need,” Advances in Neural Information Processing Systems, 2017]. Different heads of the MHSA unit process different information, which is then propagated forward to the next block after processing it through the feedforward layer, normalization layer and the residual connection. After the transformer block, a final linear layer is applied to produce the final patch embeddings. After the pre-training routine, a supervised training routine is followed, where the learned encoder from the pre-training routine is attached to multiple task heads for supervised training. The input data is modified for the training routine where instead of randomly masking the patches, mask tokens are attached at the end of the input data. For the workflow in particular, four task heads are used: Forecasting Head, Sepsis Classification Head, Shock Anomaly Detection Head, and Report Generation Head (language decoder in FIG. 6) for vitals forecasting, sepsis onset prediction, septic shock detection, and report generation, respectively.

Still referring to FIG. 3, embedded vital signs data, which have now been transformed into vectors, are then provided to various machine learning models, including a model for sepsis detection, another for vital signs forecasting, and another for septic shock prediction. Advantageously, the data vectors may be utilized to train an array of machine learning models. The CLS embedding, “<cls>emb” is used; however, other embeddings can also be employed for the classification task. Next, in the inference routine, the patient's vital signs and information are collected and encoded using the encoder; thereafter, the various task heads give the required outputs. In this routine, the mask tokens are attached towards the end of the input data, and the encoder fills in the mask tokens with forecasted patches.

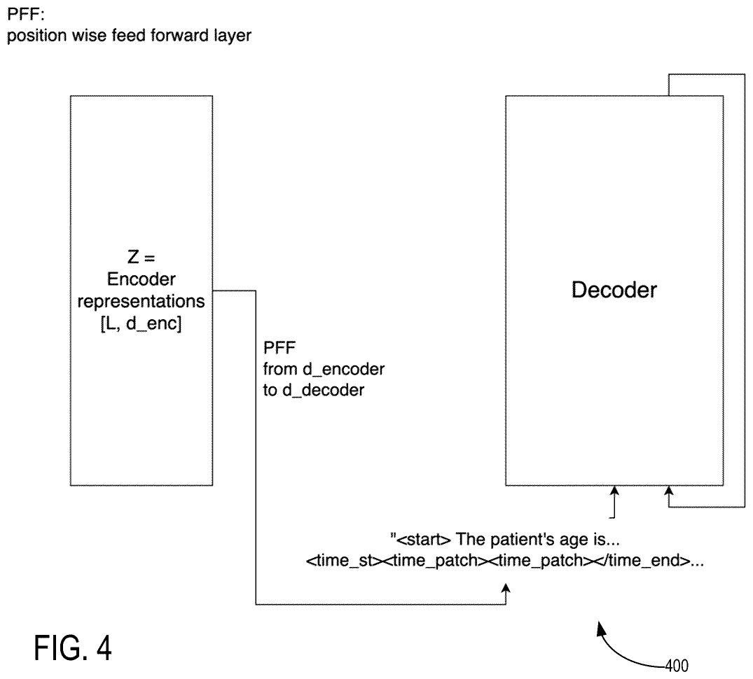

Now referring to FIG. 4, shown is a block diagram 400 illustrating a position-wise feed-forward (PFF) layer in accordance with an illustrative embodiment. This PFF layer adds non-linearity and enhances the machine learning model's ability to represent complex data relationships.

By way of example and not by way of limitation, the PFF may apply the same feed-forward neural network to each position independently.

FIG. 4 shows the pre-trained vital sign data encoder and vital sign encoded data to text decoder, i.e., a language decoder. Here, the output of the encoder is transformed using a linear projection layer, where a projection layer is a position-wise-feed-forward layer (PFF). The position-wise-feed layer is a feed-forward layer that is applied to each position separately and identically, i.e., the same weights of the feed-forward layer are applied independently to each position in the input sequence. The input of the language decoder comprises two or more elements, namely, a tokenized training prompt and projections of encoder embedding. The tokenized training prompt and projections of embeddings are concatenated before being fed to the language encoder. Parts of the training prompt can be concatenated at the beginning or end of the encoder embedding projections. The training prompt comprises additional information the language model requires to understand the patient's condition and generate a diagnostic report. The training prompt may comprise the patient's medical history, drug history and demographic information. The training prompt may also comprise special tokens to specify important information, such as “<start>” tokens to indicate the start of the training prompt, “<time_st>” and “<time_end>” tokens to indicate the start and end of time-series encoder embeddings. The language decoder is trained using a standard unsupervised pre-training routine. It is to be noted that the language decoder model can be trained from scratch or fine-tuned with a pre-trained language model.

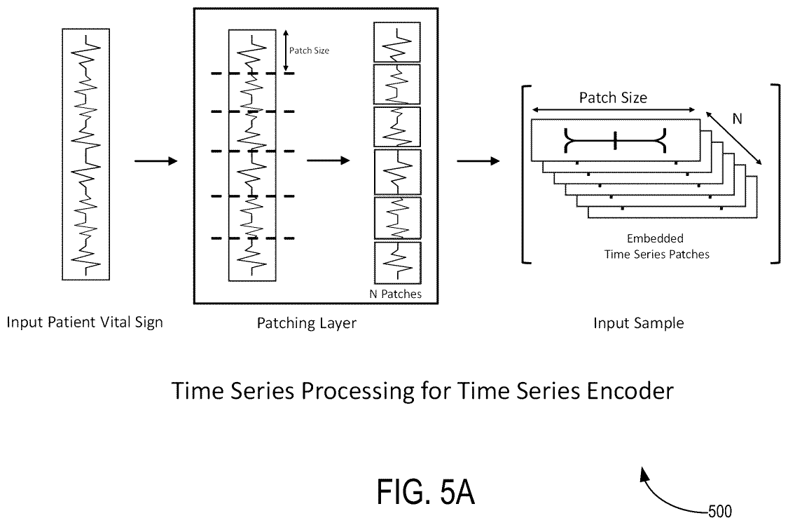

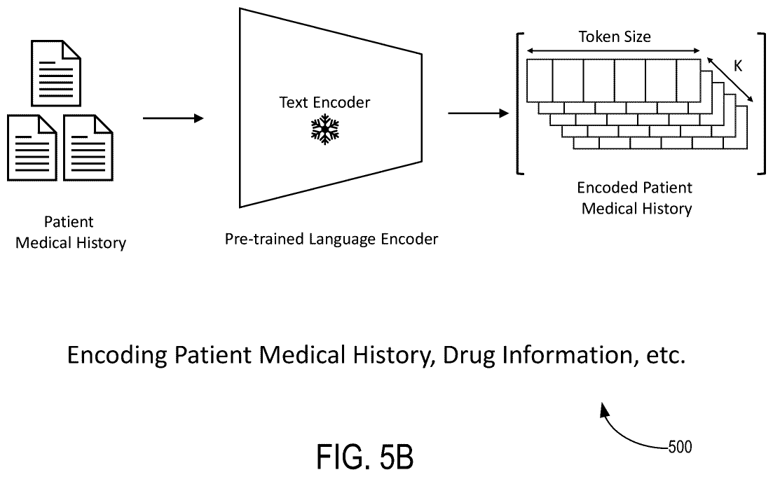

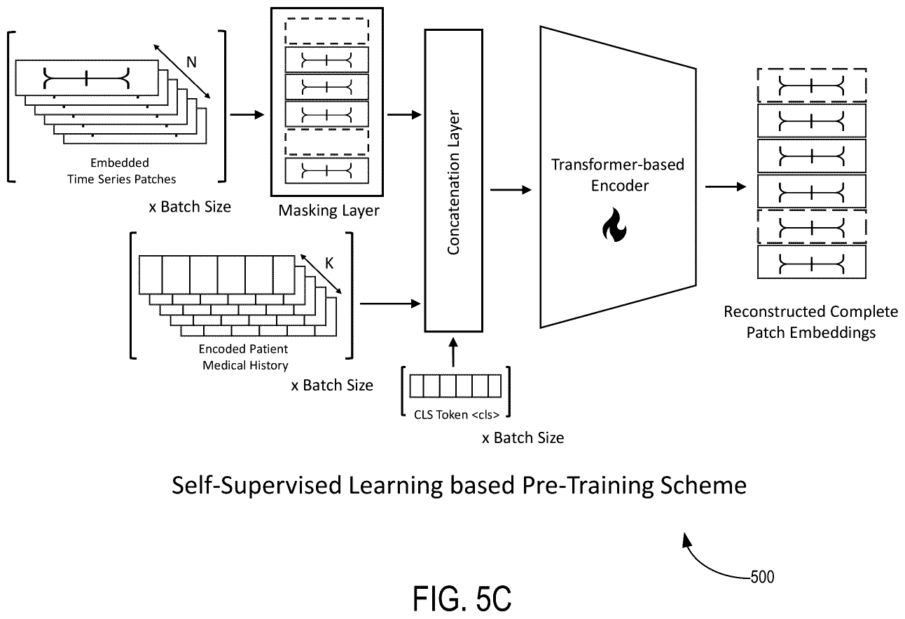

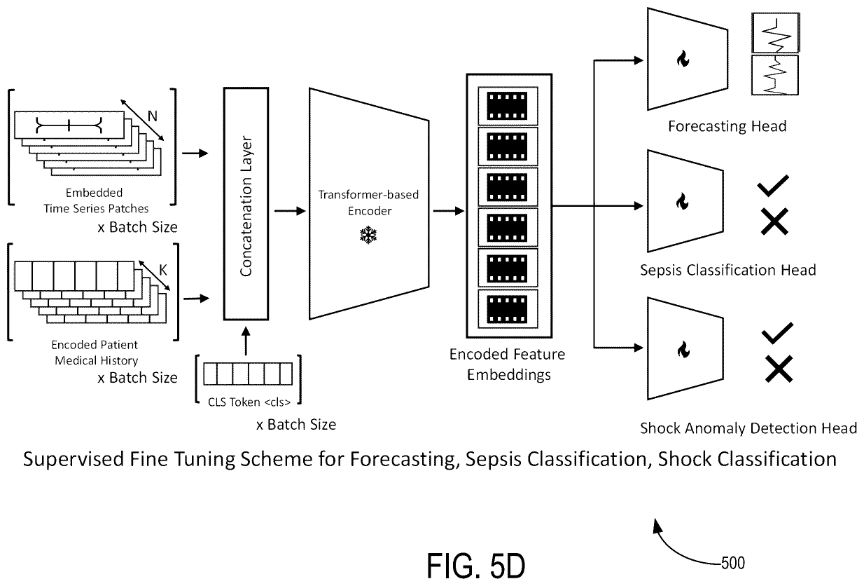

FIGS. 5A-5D and 6 show another illustrative embodiment 500. More specifically, FIG. 5A shows a schematic diagram of time series processing for a time series encoder in accordance with an illustrative embodiment. In this illustrative example, a patient vital sign is input into the system, and segmented into a plurality of segments or patches using a patching layer. In this example, the patches are of equal size and produce a set of N patches. The N patches are then used as an input sample.

Now referring to FIG. 5B, shown is a schematic diagram illustrating the encoding of a patient's medical history and other relevant information in accordance with an embodiment. In this illustrative example, a patient's medical history is provided as input into a pre-trained language encoder. Using one or more of the encoding methods discussed above, the pre-trained language encoder creates a set of K encoded patient medical history records of a particular token size.

Now referring to FIG. 5C, shown is an illustration of self-supervised learning based on a pre-training scheme. In this example, a set of embedded time series patches is processed through a masking layer and then through a concatenation layer. In this example, the masking layer allows random patches of the times series data to be masked, with the system determining what the time series data patches look like under the masks. Once trained on a sufficiently large dataset, the system is able to determine with high accuracy what a masked patch or a predicted future patch or patches would look like.

Still referring to FIG. 5C, a set of encoded patient medical history data is also provided as an input into the concatenation layer and then into a transformer-base encoder. In this example, the output from the transformer-based encoder is a set of reconstructed complete patch embeddings.

Still referring to FIG. 5C, a CLS token is also provided as an input fed to the concatenation layer, to be the concatenation layer, and then into a transformer-base encoder. In this example, the output from the transformer-based encoder is a set of reconstructed complete patch embeddings.

Now referring to FIG. 5D, shown is a schematic diagram of a supervised fine-tuning scheme for forecasting, sepsis classification and shock anomaly detection or classification in accordance with an embodiment. In FIG. 5D, the output from the transformer-based encoder is a set of encoded feature embeddings. The encoded feature embeddings at the end of the transformer encoder are dense vector representations that encapsulate the rich information derived from the input vital signs and the patient's medical history data. Here, one can use the same encoder and use different task heads in a plug-and-play manner wherein each task head is trained separately, taking the encoded feature embedding as the input and giving the desired output as required by the task. For example, a Forecasting Head in this case forecasts the patient vital signs based on the model that has been developed. A separate Sepsis Classification Head can be used to determine if Sepsis is present. A separate Shock Anomaly Detection Head can determine whether Sepsis has progressed to Septic Shock in a patient.

Now referring to FIG. 6, shown is a schematic diagram of a fine-tuning scheme for a large language model in accordance with an embodiment. In this case the encoded feature embeddings are processed through a feature space mapping step and then through a text decoder in a pre-trained LLM. The pre-trained LLM then generates a diagnostic report which summarizes all the findings and makes any recommendations.

Significantly, utilizing an integrated array of deep learning models on the patient vital signs dataset provides a more comprehensive approach to solving the complex problem of early detection of septic shock for a wide range of patients, enabling enhancing performance and extraction of nuanced insights that may not be accessible through independent models.

A significant advantage of using an integrated array of deep learning models lies in the use of the shared encoder that learns general-purpose time series representations during self-supervised pre-training rather than shallow task-specific representations. With this approach, large multi-corpus data can be used to pre-train the shared encoder, which enables us to encode vital sign data into rich feature embeddings. As a result, the learned representations generalize better across a wide range of tasks without needing task-specific tuning or with minimal task-specific tuning. One can use the same encoder and use different task heads in a plug-and-play manner wherein each task head is trained separately, taking the encoded feature embedding as the input and giving the desired output as required by the task. This is in contrast to having separate models for specific tasks, wherein an entire deep neural network must be trained from scratch to achieve a single task.

A first advantage to using an integrated array of models is the ability to adopt diverse strategies for problem solving. For example, utilizing the same dataset, different types of deep leaning models may be used to perform analysis on different aspects of a patient's vital data. For instance, convolutional neural networks (CNNs) are adept at identifying spatial hierarchies in images, while recurrent neural networks (RNNs) excel at processing sequential data. An integrated approach can leverage the strengths of different machine learning models simultaneously, and to cross-reference the results of each model.

Furthermore, a combination of different deep learning models can reduce the likelihood of overfitting a model to noise in the dataset, as different models may overfit to different aspects, thereby increasing the robustness of predictions or analyses. In particular, for projecting patient vital signs data over the next several hours for early prediction of septic shock, different types of analysis for different aspects of the patient vital signs data may be used for the prediction. Furthermore, if the patient is categorized into one of a number of different categories, a machine learning model which has been specifically trained on data belonging to the same category of patient may potentially provide a more accurate prediction.

In addition, as different models may extract and learn from different features within the same dataset, a multi-faceted view can be obtained which may lead to a more holistic understanding of the data. Combining predictions from multiple models (ensemble methods) may thus result in higher accuracy than any single model could achieve on its own, and this ensemble consensus approach may mitigate against individual errors.

Furthermore, as models are trained simultaneously on the same dataset, parallel processing techniques can be utilized to potentially speeding up the training and prediction processes. By analyzing how different models interact with the same dataset, the present system and method may result in a more robust way to accurately predict septic shock.

Advantageously, by using an integrated array of machine learning models, the present system and method can transform patient vital signs data into actionable insights for healthcare providers through a comprehensive dashboard or report output. By aggregating data from diverse sources, including real time patient vital signs data, electronic health records, wearable devices, and clinical tests, these models can analyze patterns and trends across various health metrics. Thus, the dashboard synthesizes the analyses performed by the models, presenting them in an intuitive and accessible format. It highlights key indicators, such as risk levels for specific conditions, changes in vital signs, and recommendations for further tests or interventions. This allows healthcare providers to quickly assess a patient's health status, monitor progress over time, and make informed decisions on treatment plans. The integration of these models into a single dashboard ensures a holistic view of patient health, enabling personalized and timely care.

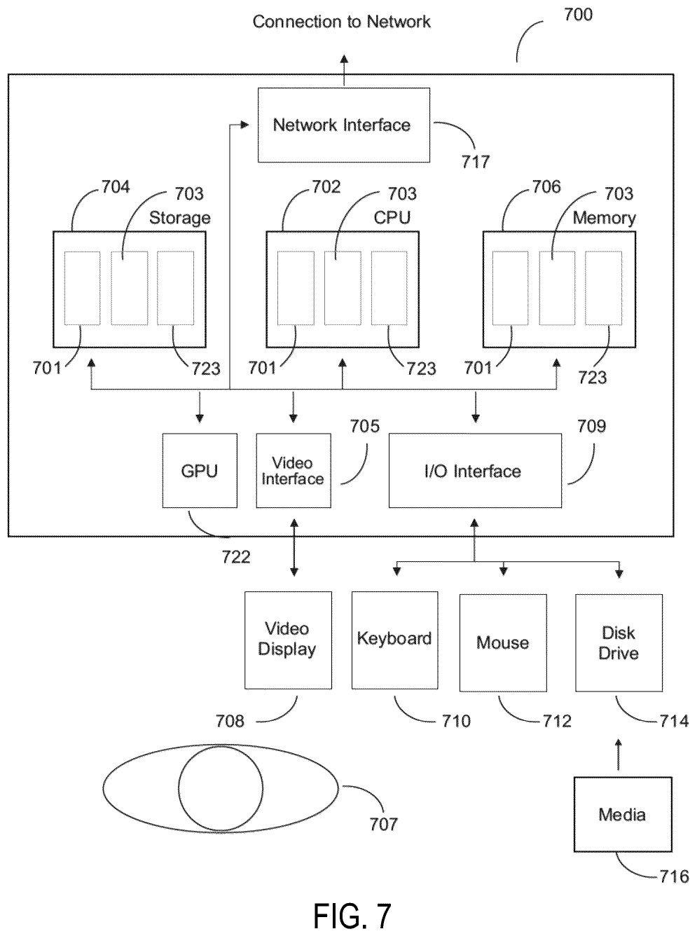

Now referring to FIG. 7, shown is a schematic block diagram of a generic computing device that may provide a suitable operating environment in one or more embodiments. A suitably configured computer device, and associated communications networks, devices, software, and firmware may provide a platform for enabling one or more embodiments as described above. By way of example, FIG. 5 shows a generic computer device 700 that may include a central processing unit (“CPU”) 702 connected to a storage unit 704 and to a random-access memory 706. The CPU 702 may process an operating system 701, application program 703, and data 723. The operating system 701, application program 703, and data 723 may be stored in storage unit 704 and loaded into memory 706, as may be required. Computer device 700 may further include a graphics processing unit (GPU) 722 which is operatively connected to CPU 702 and to memory 706 to offload intensive image processing calculations from CPU 702 and run these calculations in parallel with CPU 702. An operator 710 may interact with the computer device 700 using a video display 708 connected by a video interface 705, and various input/output devices such as a keyboard 710, pointer 712, and storage 714 connected by an I/O interface 709. In known manner, the pointer 712 may be configured to control movement of a cursor or pointer icon in the video display 708, and to operate various graphical user interface (GUI) controls appearing in the video display 708. The computer device 700 may form part of a network via a network interface 711, allowing the computer device 700 to communicate with other suitably configured data processing systems or circuits. A non-transitory medium 716 may be used to store executable code embodying one or more embodiments of the present method on the generic computing device 700.

### Parameter-Efficient Fine-Tuning (PEFT)

In another embodiment, rather than conducting pre-training of the encoder on a large corpus of unlabeled data through Self-Supervised Learning (SSL), a subset of weights within a pre-trained large time-series encoder may be selectively fine-tuned on domain-specific data, such as physiological vital signs. A method for embedding domain-specific knowledge into the encoder comprises the application of Parameter-Efficient Fine-Tuning (PEFT) techniques. Following this approach, a minimal subset of weights, pre-existing within the encoder architecture or newly introduced, is fine-tuned, while the remainder of the model weights are frozen. This method leverages the robust feature representations acquired during initial pre-training while allowing for the incorporation of domain-specific adaptations through selective fine-tuning. As a result, the computational requirements and inefficiencies associated with fine-tuning the entire encoder on a specific task or dataset are significantly reduced. Described below are two PEFT techniques, though the scope of this embodiment is not limited to these methods.

Selective PEFT: Selective PEFT provides domain adaptation by fine-tuning only a small set of model weights already in the architecture. For the experiments, the following techniques were used:
1. BitFit: This technique focuses on fine-tuning only the bias terms within the model architecture. By adjusting these bias parameters, BitFit can achieve task/data-specific adaptations with minimal changes to the overall model structure.
2. LayerNorm (LN) Tuning: This technique fine-tunes the parameters of the LN components present in the model architecture's attention module. By adjusting the weights and biases of LN layers, the model can better normalize activations across different tasks, potentially stabilizing training and improving performance.

Additive PEFT: Additive PEFT enables domain adaptation by introducing new parameters into the model architecture, which are then fine-tuned while keeping all the original weights frozen. For the experiments, the following techniques were used:
1. Vector-based Random Matrix Adaptation (VeRA): This technique adds a parallel processing path in the linear layers of the attention module using fixed, randomly initialized rank matrices, similar to Low-Rank Adaptation (LoRA). These matrices are shared across layers and remain frozen during training. VeRA introduces two learnable scaling vectors that facilitate domain adaptation when multiplied by the frozen matrices. The weight update is given by:

$${W}^{′} = W + {λ}_{b} B {λ}_{d} A$$

Here, W is the original weight matrix, W^{∧}′ is the updated weight matrix, B and A are the frozen rank matrices, and λ_b and λ_d are the learnable scaling vectors (used as diagonal matrices) for matrices B and A respectively, that modulate the adaptation.
2. Fourier Transform for Fine-Tuning (FourierFT): This technique leverages the Fourier transform to reduce the number of trainable parameters by encoding them into a small set of spectral coefficients. These coefficients correspond to a subset of spectral entries randomly initialized and shared across all layers. The inverse discrete Fourier transform (IDFT) is then applied to convert the modified spectral data back into the spatial domain, forming the weight update matrix. The weight update in FourierFT can be expressed as:

$${W}^{′} = W + αℜ \{ {F}^{- 1} ( E ⊙ c ) \} ,$$

where E represents the frozen spectral entries, c is the set of learnable spectral coefficients, ⊙ denotes the element-wise operation to create the spectral matrix, [symbol] ⁻¹ is the IDFT, [symbol] extracts the real part of the transformed data, and a serves as a scaling factor.

Foundational Models (FMs) have advanced deep learning by offering robust, pre-trained architectures effective across multiple domains. Inspired by their success in language and vision, researchers have begun exploring Time Series FMs (TSFMs) to address the complexities of large-scale time series data, such as non-standard quantization, varying scales and frequencies, and irregular intervals. While TSFMs perform well on datasets similar to their pre-training data (e.g., retail, finance, transport, weather), they underperform in domains with scarce publicly available data like healthcare. Healthcare data involves diverse signals and tasks—including stress detection, emotion recognition, vitals forecasting and others in both contact and non-contact settings. Although task-specific models exist, they lack the generalized representations learned by large FMs. Recent efforts to develop domain-specific TSFMs, such as SleepFM and ECG-FM, have resulted in highly specialized models which are, however, less adaptable to a broader range of tasks.

A common method of integrating domain knowledge into FMs is through Parameter-Efficient Fine-Tuning (PEFT), which is widely used in language and vision FMs. These techniques fine-tune only a small subset of weights—either selected from the existing FM architecture or newly introduced, while keeping the rest of the model weights frozen. This approach leverages the rich feature representations learned by FMs during pre-training while integrating domain-specific knowledge through targeted fine-tuning. By doing so, it reduces the computational burden and inefficiencies associated with fine-tuning the entire FM for a specific task or dataset. However, the impact of PEFT on TSFMs for out-of-domain data remains largely unexplored. A recent study by provides insights into the application of Low-Rank Adaptation (LoRA), a prevalent PEFT technique, within TSFMs in the healthcare domain. There are, however, more recent and efficient PEFT techniques that claim to achieve comparable, if not superior, performance to LoRA while fine-tuning even fewer parameters. This is particularly advantageous in scenarios with limited datasets, how-ever they are yet to be applied and benchmarked in the context of TSFMs. To address this gap, several other PEFT techniques were introduced into TSFMs for forecasting vital signs of sepsis patients in critical care and conduct a comparative analysis. In summary, the following steps were undertaken.
1. Present and assess two selective and two additive PEFT methods, thereby broadening the spectrum of fine-tuning approaches explored with multiple configurations of the Chronos TSFM.
2. Perform a thorough comparative analysis of these PEFT methods against LoRA, high-lighting their influence on performance and parameter efficiency in the context of forecasting vital signs, a task involving out-of-domain modalities for TSFMs.
3. Benchmark findings against state-of-the-art (SOTA) models that are trained from scratch with a large number of parameters. The results demonstrate that PEFT can enable certain TSFM variants to achieve, and in some cases exceed, the performance of these models while requiring fine-tuning of significantly fewer parameters.

### Methodology

In this section, a brief overview of the methodology is provided, which includes the TSFM used as the backbone for these experiments, the two selective and two additive PEFT methods, the dataset and its preprocessing, and finally, the implementation details.

Backbone: Recently, multiple TSFMs have been introduced, each employing different strategies for modeling time series data. Among these, the Chronos family of TSFMs is used in these experiments due to their superior performance across various settings—including zero-shot, fully fine-tuned, and LoRA fine-tuned settings. Chronos utilizes the encoder-decoder transformer architecture from the T5 language FM family to perform time series forecasting. The model simplifies data processing by discretizing real-valued inputs through a binning and scaling function. Additionally, Chronos offers multiple-sized model variants providing valuable insights into the effects of scaling when introducing PEFT on larger FMs.

Selective PEFT: Selective PEFT provides domain adaptation by fine-tuning only a small set of model weights already present in the architecture. For these experiments, the following techniques were used:
BitFit. This technique focuses on fine-tuning only the bias terms within the model architecture. By adjusting these bias parameters, BitFit can achieve task/data-specific adaptations with minimal changes to the overall model structure.
LayerNorm (LN) Tuning. This technique fine-tunes the parameters of the LN components present in the attention module of the model architecture. By adjusting the weights and biases of LN layers, the model can learn to better normalize activations across different tasks, potentially stabilizing training and improving performance.

Additive PEFT: Additive PEFT enables domain adaptation by introducing new parameters into the model architecture, which are then fine-tuned keeping all the original weights frozen. For these experiments, the following techniques were used:
Vector-based Random Matrix Adaptation (VeRA). This technique adds a parallel processing path in the linear layers of the attention module using fixed, randomly initialized rank matrices, similar to LoRA. These matrices are shared across layers and remain frozen during training. VeRA introduces two learnable scaling vectors that, when multiplied by the frozen matrices, facilitate domain adaptation. The weight update is given by:

$${W}^{′} = W + {λ}_{b} {Bλ}_{d} A .$$

Here, W is the original weight matrix, W.' is the updated weight matrix, B and A are the frozen rank matrices, and λ_{b} and λ_{d} are the learnable scaling vectors (used as diagonal matrices) for matrices B and A respectively that modulate the adaptation.
Fourier Transform for Fine-Tuning (FourierFT). This technique leverages the Fourier transform to reduce the number of trainable parameters by encoding them into a small set of spectral coefficients. These coefficients correspond to a subset of spectral entries, which are randomly initialized and shared across all layers. The inverse discrete Fourier transform (IDFT) is then applied to convert the modified spectral data back into the spatial domain, forming the weight update matrix. The weight update in FourierFT can be expressed as:

$${W}^{′} = W + aR \{ {F}^{( - 1 )} ( E ⊙ c ) \}$$

where E represents the frozen spectral entries, c is the set of learnable spectral coefficients, ⊙ denotes the element-wise operation to create the spectral matrix, F⁽⁻¹⁾ is the IDFT, R extracts the real part of the transformed data, and a serves as a scaling factor.

TABLE 1
Results compare MeanBP and HR forecasts across various fine-tuning settings, including full fine-
tuning (FullFT) as reported in. The system and method also analyzes the total number of trainable
parameters (in millions) to assess efficiency. The best results for each fine-tuning setting
within each TSFM are underlined, while the overall best values are highlighted in bold.

| | | MeanBP* | | | HR* | | | #Params. |
|---|---|---|---|---|---|---|---|---|
| Model | Setting | MSE ↓ | DTW ↓ | MAPE ↓ | MSE ↓ | DTW ↓ | MAPE ↓ | (in M.) ↓ |
| Bhatti et | N-HiTS | 19.81 | 16.32 | 7.90 | 7.18 | 7.92 | 6.27 | 0.7 |
| al. [8] | N-BEATS | 27.42 | 18.60 | 9.64 | 12.98 | 17.90 | 9.15 | 0.73 |
| | TFT | 19.00 | 23.46 | 7.78 | 7.57 | 15.79 | 6.52 | 0.53 |
| Chronos | 0-shot | 25.60 | 21.40 | 8.64 | 7.37 | 11.41 | 5.97 | — |
| (Tiny) | Full FT [4] | 19.90 | 20.51 | 8.03 | 8.80 | 14.99 | 6.86 | 8.3 |
| | LoRA [4] | 19.79 | 19.86 | 7.90 | 7.22 | 11.17 | 5.90 | 0.049 |
| | BitFit | 20.68 | 19.79 | 8.01 | 7.35 | 11.31 | 5.95 | 0.0002 |
| | LN Tuning | 20.25 | 19.05 | 7.87 | 7.38 | 11.30 | 5.94 | 0.005 |
| | VeRA | 20.16 | 18.50 | 7.80 | 7.29 | 11.19 | 5.96 | 0.013 |
| | FourierFT | 19.51 | 18.55 | 7.76 | 7.06 | 10.80 | 5.81 | 0.002 |
| Chronos | 0-shot | 25.04 | 20.28 | 8.49 | 7.19 | 11.01 | 5.92 | — |
| (Small) | Full FT [4] | 20.93 | 20.95 | 8.23 | 10.04 | 16.53 | 7.37 | 46.1 |
| | LoRA [4] | 19.89 | 20.44 | 8.02 | 7.08 | 10.83 | 5.88 | 0.147 |
| | BitFit | 20.65 | 19.87 | 8.09 | 7.19 | 10.90 | 5.93 | 0.0005 |
| | LN Tuning | 19.81 | 19.85 | 7.99 | 7.21 | 10.88 | 5.94 | 0.016 |
| | VeRA | 20.98 | 19.08 | 7.98 | 7.21 | 10.87 | 5.94 | 0.038 |
| | FourierFT | 19.65 | 19.98 | 7.94 | 7.20 | 10.82 | 5.93 | 0.003 |
| Chronos | 0-shot | 25.70 | 20.32 | 8.53 | 7.33 | 11.02 | 5.96 | — |
| (Base) | Full FT [4] | 20.80 | 21.09 | 8.20 | 10.15 | 16.82 | 7.37 | 201 |
| | LoRA [4] | 20.12 | 21.06 | 8.06 | 7.25 | 10.96 | 5.93 | 0.442 |
| | BitFit | 19.77 | 19.30 | 7.91 | 7.42 | 11.16 | 5.99 | 0.0007 |
| | LN Tuning | 19.87 | 19.58 | 7.92 | 7.41 | 11.16 | 5.99 | 0.047 |
| | VeRA | 21.58 | 18.30 | 7.92 | 7.40 | 11.10 | 5.97 | 0.113 |
| | FourierFT | 20.98 | 17.96 | 7.86 | 7.40 | 11.12 | 5.97 | 0.007 |

*MSE values are normalized by 1e−4, and DTW values by 1e−3 for clearer interpretation.

Dataset and Processing: The system and method may use the publicly available elCU Collaborative Research Database to forecast mean blood pressure (MeanBP) and heart rate (HR) for sepsis patients in ICU. Following methods from, missing values were imputed with forward filling. 9-hour windows of vital signs were extracted before diagnosis, with the first 6 hours as context and the last 3 hours as the prediction horizon. Vitals were sampled every 5 minutes, resulting in context and horizon windows of 72 and 36, respectively. A low-pass filter was then applied to reduce noise, followed by global min-max scaling for normalization. The preprocessed dataset consisted of 4,020 samples from 1,442 patients, split into training, validation, and test sets in an 8:1:1 ratio, with no patient data overlapping across splits.

Implementation Details: Performance was optimized across various data types and fine-tuning con-figurations using learning rates between 1e-2 and 1e-5 with the Adam optimizer. For additive PEFT methods, all attention weight matrices (Query, Key, Value, and Output) were fine-tuned for both FourierFT and VeRA for fair comparison with LoRA used in. For VeRA, the adapter rank r was set to 16, which is much higher than the rank 2 used for LoRA in prior work, following recommendations due to the smaller fraction of fine-tuned weights. For FourierFT, a was set to 300, with number of coefficients, n tuned to 50. A rationale for selecting the above values for r and n in provided below. Other hyperparameters were kept as specified in the original model check-points. Experiments were conducted in PyTorch on an NVIDIA RTX 4090 GPU, with forecasting performance evaluated using mean squared error (MSE), dynamic time warping (DTW), and mean average percentage error (MAPE). Owing to the probabilistic nature of the forecasts, the median of 20 samples is used for each prediction, with metrics averaged over 10 runs for robust evaluation.

The system and method evaluates the effectiveness of various PEFT techniques across different Chronos model variants—Tiny, Small, and Base—comprising 8.3M, 46.1M, and 201M trainable parameters, respectively, with the results presented in Table 1. These findings are further compared against LoRA and full model fine-tuning as reported in. It was observed that, in nearly all configurations, the PEFT methods consistently outperform the TSFM without domain adaptation (zero-shot) and full fine-tuning approaches. Techniques such as BitFit, LN Tuning, VeRA, and FourierFT demonstrate comparable or superior performance to LoRA across multiple metrics for both MeanBP and HR forecasting, while requiring significantly fewer trainable parameters. Notably, FourierFT, when applied to the Chronos (Tiny) variant, surpasses the SOTA model. Here, the SOTA model comprises approximately 700 K trainable parameters, whereas FourierFT requires fine-tuning of only 2,400 parameters. This highlights the effectiveness of training a small set of parameters for domain adaptation, leveraging the strong generalization capabilities of large FMs, rather than training smaller models with a narrow focus on specific data or tasks.

Among all the PEFT techniques, BitFit fine-tuned the smallest number of parameters while delivering comparable and sometimes even the best performance. For example, in the case of Chronos (Base), which originally has 201M parameters, BitFit fine-tuned only 768 parameters and achieved the best values for the MSE metric in MeanBP forecasting. It closely followed FourierFT, the best-performing PEFT in that setting, which fine-tuned 7200 parameters. After BitFit, FourierFT consistently gave the best results for both vitals across multiple metrics in various settings. It ex-celled particularly with the other two TSFMs particularly with Chronos (Tiny) which surpassed the SOTA by fine-tuning a small number of parameters compared to techniques like LORA, VeRA, and LN Tuning. This aligns with the findings of the original work, where the authors propose that only a small number of sparse coefficients need to be learned in the spectral domain to achieve the desired effect in the spatial domain. LN Tuning required more fine-tunable parameters com-pared to FourierFT, but still produced comparable results in several settings. Among the new PEFT techniques introduced, VeRA fine-tuned the most parameters, approaching the parameter count of LORA. This can be explained by the way PEFT is performed in VeRA. Since VeRA fine-tunes only the scaling vectors, the rank of the frozen matrices needs to be much higher than in LoRA to achieve similar results. For instance, while LoRA used rank-2 matrices to fine-tune 49K parameters for Chronos (Tiny), VeRA required rank-16 matrices to fine-tune 13K parameters to attain comparable performance. Interestingly, it was observed that for Chronos (Base) using LoRA with 442K trainable parameters yields the best performance for forecasting HR vitals. This can be attributed to the relatively low variability in HR readings, which likely necessitate tuning a larger number of parameters in a model of this size (201M original parameters) that has been pre-trained on a broad range of highly variable time series data.

Overall, the results underscore a critical trade-off between the number of fine-tuned parameters and model performance. While techniques such as BitFit and FourierFT demonstrate that competitive outcomes can be achieved by fine-tuning a minimal subset of parameters, methods like VeRA and LN Tuning involve fine-tuning a larger number of parameters, which can lead to improved performance in certain settings but at the cost of increased computational requirements. Moreover, additive methods like FourierFT, VeRA, and LoRA offer greater flexibility, owing to the additional hyperparameters that accompany these techniques.

A comparative study of various PEFT techniques was conducted within the context of adapting TSFMs for forecasting healthcare time series data, specifically vital signs in ICUs. Two selective PEFT methods were explored, BitFit and LayerNorm Tuning, alongside two additive PEFT methods, VeRA and FourierFT. The results showed that in addition to the commonly used LoRA method, other PEFT techniques achieve comparable or superior performance, fine-tuning only a small fraction of the total model parameters. Notably, FourierFT, when applied to Chronos (Tiny) TSFM, outperformed SOTA models trained from scratch on this task. These experiments further demonstrated that tuning as few as 256 parameters (BitFit) can yield competitive results underscoring the importance of continued research in the evolving TSFM space.

### Additional Experiments

This section describes supplementary experiments that were undertaken. These include results from tuning the hyperparameters of additive PEFT techniques, and also the experiment results from the other two Chronos variants (Mini—20.4M parameters and Large—708M parameters) for comprehensiveness.

### VeRA:

As shown in Table 2, below, setting the rank r to 16 provided the optimal results when tuning this hyperparameter. Additionally, as r increased, the change in the number of tunable parameters was not substantial. This is due to VeRA's approach, where instead of learning full matrices, the PEFT learns only scaling vectors, significantly reducing the parameter overhead despite higher ranks.

TABLE 2
Results comparing MeanBP and HR forecasts across different ranks
r of the introduced adapters by VeRA for adapting Chronos (Tiny).

| | MeanBP* | | | HR* | | | #Params. |
|---|---|---|---|---|---|---|---|
| Rank (r) | MSE ↓ | DTW ↓ | MAPE ↓ | MSE ↓ | DTW ↓ | MAPE ↓ | (in M.) ↓ |
| 1 | 20.43 | 21.31 | 8.16 | 7.36 | 11.31 | 5.99 | 0.0123 |
| 2 | 20.20 | 20.76 | 8.06 | 7.30 | 11.20 | 5.96 | 0.0124 |
| 4 | 20.10 | 18.65 | 7.87 | 7.31 | 11.18 | 5.96 | 0.0125 |
| 8 | 20.25 | 18.59 | 7.87 | 7.32 | 11.20 | 5.96 | 0.0127 |
| 16 | 20.16 | 18.50 | 7.80 | 7.29 | 11.19 | 5.96 | 0.0131 |
| 32 | 20.26 | 18.59 | 7.87 | 7.35 | 11.24 | 5.96 | 0.0139 |

*MSE values are normalized by 1e−4, and DTW values by 1e−3 for clearer interpretation.

### FourierFT

Similar to VeRA, Table 3 below shows that setting the number of spectral coefficients n to 50 yielded the optimal results when tuning this hyperparameter. This configuration consistently provided the best performance across the evaluated settings.

TABLE 3
Results comparing MeanBP and HR forecasts across different
number of spectral coefficients n of the introduced
adapters by FourierFT for adapting Chronos (Tiny).

| #Spectral | MeanBP* | | | HR* | | | #Params. |
|---|---|---|---|---|---|---|---|
| Coefficients (n) | MSE ↓ | DTW ↓ | MAPE ↓ | MSE ↓ | DTW ↓ | MAPE ↓ | (in M.) ↓ |
| 25 | 19.89 | 18.67 | 7.85 | 7.17 | 10.98 | 5.88 | 0.0012 |
| 50 | 19.51 | 18.55 | 7.76 | 7.06 | 10.80 | 5.81 | 0.0024 |
| 100 | 19.71 | 20.44 | 7.93 | 7.31 | 11.19 | 5.99 | 0.0048 |
| 200 | 19.85 | 20.46 | 7.99 | 7.39 | 12.01 | 6.16 | 0.0096 |

*MSE values are normalized by 1e−4, and DTW values by 1e−3 for clearer interpretation.

### Chronos

Table 4 below shows the results of experimenting with the remaining Chronos variants, where similar trends to those discussed above with reference to Section 3 are observed. For MeanBP, VeRA, and FourierFT outperformed LoRA on DTW and MAPE metrics. However, for the lower variability vital, HR forecasting, LoRA remained the best-performing PEFT method. Additionally, for Chronos (Large), fine-tuning fewer parameters than LoRA did not lead to better performance with results stagnating regardless of the number of parameters fine-tuned, likely due to the large-scale nature of the model. FourierFT, however, stood as an exception, yielding distinct outcomes compared to the other newly introduced PEFT methods. This divergence can be attributed to FourierFT's unique processing and learning mechanisms in the spectral domain.

TABLE 4
Results comparing MeanBP and HR forecasts across different fine-tuning settings
including full fine-tune (FullFT) from [4] for Chronos (Mini) and Chronos
(Large). The best values for the fine-tune settings are underlined for each TSFM.

| | | MeanBP* | | | HR* | | | #Params. |
|---|---|---|---|---|---|---|---|---|
| Model | Setting | MSE ↓ | DTW ↓ | MAPE ↓ | MSE ↓ | DTW ↓ | MAPE ↓ | (in M.) ↓ |
| Chronos | 0-shot | 25.21 | 20.44 | 8.52 | 7.44 | 11.20 | 5.97 | — |
| (Mini) | Full FT [4] | 20.50 | 21.80 | 8.19 | 10.46 | 17.45 | 7.50 | 20.4 |
| | LoRA [4] | 20.05 | 20.72 | 8.03 | 7.26 | 10.88 | 5.90 | 0.086 |
| | BitFit | 20.68 | 20.02 | 8.01 | 7.47 | 11.25 | 5.98 | 0.0005 |
| | LN Tuning | 20.48 | 21.26 | 8.19 | 7.48 | 11.22 | 5.99 | 0.008 |
| | VeRA | 20.21 | 17.95 | 7.73 | 7.44 | 11.19 | 5.97 | 0.024 |
| | FourierFT | 20.43 | 21.46 | 8.17 | 7.45 | 11.10 | 5.97 | 0.002 |
| Chronos | 0-shot | 25.54 | 19.75 | 8.50 | 7.21 | 10.84 | 5.93 | — |
| (Large) | Full FT [4] | 21.01 | 20.58 | 8.22 | 9.56 | 16.45 | 7.32 | 708 |
| | LoRA [4] | 20.00 | 20.34 | 8.06 | 7.16 | 10.76 | 5.91 | 1.1 |
| | BitFit | 25.09 | 19.49 | 8.43 | 7.30 | 10.92 | 5.94 | 0.001 |
| | LN Tuning | 25.09 | 19.49 | 8.43 | 7.30 | 10.92 | 5.94 | 0.125 |
| | VeRA | 25.09 | 19.49 | 8.43 | 7.30 | 10.92 | 5.94 | 0.3 |
| | FourierFT | 22.11 | 18.24 | 7.97 | 7.31 | 10.91 | 5.94 | 0.014 |

*MSE values are normalized by 1e−4, and DTW values by 1e−3 for clearer interpretation.

In summary, using Parameter-Efficient Fine-Tuning (PEFT) techniques to selectively fine-tune a subset of weights within a pre-trained large time-series encoder on domain-specific data (such as medical) has been demonstrated to be promising, and providing a system and method for incorporating these additional modeling tools can provide additional options for modeling patient vital signs into the future.

Thus, in an aspect, there is provided a method of early prediction of sepsis or septic shock through bio-data analysis on a computing device, the method comprising: training one or more time series deep learning models by: encoding sepsis or septic shock patient vital signs as time series-based bio-data to generate embedded time series patches; encoding associated patient medical histories utilizing a pre-trained language encoder to generate encoded patient medical history patches; combining the embedded time series patches and the patient medical history patches into an array; and randomly masking the embedded time series patches and asking the model to reconstructing the masked patches based at least in part on the associated patient medical histories; and utilizing the one or more trained time series deep learning models to predict sepsis or septic shock for a new patient.

In an embodiment, the method further comprises conducting a supervised training routine for one or more task heads layered on top of the one or more trained time series deep learning models, the one or more task heads comprising a vital signs forecasting head, a sepsis classification head, a shock anomaly detection head, and a report generation head.

In another embodiment, the supervised training routine comprises utilizing masked patches to make predictions about the patient's vital signs, and utilizing a learnable classification token to classify sepsis or septic shock.

In another embodiment, the supervised training for the report generation head comprises utilizing feature space mapping to generate a report comprising the predictions about the patient's vital signs, whether the patient is classified as having sepsis or septic shock, a summary of the patient's current medical condition, and recommendations for treatment and an action plan.

In another embodiment, vital signs data for training the time series deep learning models is encoded using one or more of Min-Max scaling, lagged features, statistical features, a Fourier transform of input vital signs, to encode information in the frequency domain, one-hot encoding, and ordinal encoding.

In another embodiment, the method further comprises presenting the output of the time series deep learning models in an integrated early warning dashboard.

In another embodiment, providing a reinforcement learning agent which can use this human feedback on the recommended diagnostic and treatment plans to improve the future response of the Generative AI model.

In another embodiment, the time series deep learning models comprise one or more time series foundation models, and the method further comprises using Parameter-Efficient Fine-Tuning techniques to fine tune selected trainable parameters of these models for healthcare applications.

In another embodiment, the method further comprises training a time series foundation model utilizing one or more Parameter-Efficient Fine-Tuning techniques comprising LoRA, VeRA, FourierFT.

In another embodiment, the vital signs forecasting head is adapted to forecast the patient vital signs into the future to predict sepsis or septic shock for the new patient.

In another aspect, there is provided a system for early prediction of sepsis or septic shock through bio-data analysis on a computing device, the system adapted to: train one or more time series deep learning models by: encoding sepsis or septic shock patient vital signs as time series-based bio-data to generate embedded time series patches; encoding associated patient medical histories utilizing a pre-trained language encoder to generate encoded patient medical history patches; combining the embedded time series patches and the patient medical history patches into an array; and randomly masking the embedded time series patches and asking the model to reconstructing the masked patches based at least in part on the associated patient medical histories; and utilize the one or more trained time series deep learning models to predict sepsis or septic shock for a new patient.

In an embodiment, the system is further adapted to: conduct a supervised training routine for one or more task heads layered on top of the one or more trained time series deep learning models, the one or more task heads comprising a vital signs forecasting head, a sepsis classification head, a shock anomaly detection head, and a report generation head.

In another embodiment, the supervised training routine comprises utilizing masked patches to make predictions about the patient's vital signs, and utilizing a learnable classification token to classify sepsis or septic shock.

In another embodiment, the supervised training for the report generation head comprises utilizing feature space mapping to generate a report comprising the predictions about the patient's vital signs, whether the patient is classified as having sepsis or septic shock, a summary of the patient's current medical condition, and recommendations for treatment and an action plan.

In another embodiment, vital signs data for training the time series deep learning models is encoded using one or more of Min-Max scaling, lagged features, statistical features, a Fourier transform of input vital signs, to encode information in the frequency domain, one-hot encoding, and ordinal encoding.

In another embodiment, the system if further adapted to present the output of the time series deep learning models in an integrated early warning dashboard.

In another embodiment, the system if further adapted to provide a reinforcement learning agent which can use this human feedback on the recommended diagnostic and treatment plans to improve the future response of the Generative AI model.

In another embodiment, time series deep learning models comprise one or more time series foundation models, and the method further comprises using Parameter-Efficient Fine-Tuning techniques to fine tune selected trainable parameters of these models for healthcare applications.

In another embodiment, the system is further adapted to train a time series foundation model utilizing one or more Parameter-Efficient Fine-Tuning techniques comprising LoRA, VeRA, FourierFT.

In another embodiment, the vital signs forecasting head is adapted to forecast the patient vital signs into the future to predict sepsis or septic shock for the new patient.

Although exemplary embodiments of the present invention has been described with reference to the accompanying drawings, those skilled in the art to which the present disclosure belongs will appreciate that various modifications and alterations may be made without departing from the spirit or essential feature of the present invention. Therefore, it is to be understood that embodiments described above are illustrative rather than being restrictive in all aspects.

1. A method of early prediction of sepsis or septic shock through bio-data analysis on a computing device, the method comprising:
training one or more time series deep learning models by:
encoding sepsis or septic shock patient vital signs as time series-based bio-data to generate embedded time series patches;
encoding associated patient medical histories utilizing a pre-trained language encoder to generate encoded patient medical history patches;
combining the embedded time series patches and the patient medical history patches into an array; and
randomly masking the embedded time series patches and asking the model to reconstructing the masked patches based at least in part on the associated patient medical histories; and
utilizing the one or more trained time series deep learning models to predict sepsis or septic shock for a new patient.

2. The method of claim 1, further comprising:
conducting a supervised training routine for one or more task heads layered on top of the one or more trained time series deep learning models, the one or more task heads comprising a vital signs forecasting head, a sepsis classification head, a shock anomaly detection head, and a report generation head.

3. The method of claim 2, wherein the supervised training routine comprises utilizing masked patches to make predictions about the patient's vital signs, and utilizing a learnable classification token to classify sepsis or septic shock.

4. The method of claim 3, wherein the supervised training for the report generation head comprises utilizing feature space mapping to generate a report comprising the predictions about the patient's vital signs, whether the patient is classified as having sepsis or septic shock, a summary of the patient's current medical condition, and recommendations for treatment and an action plan.

5. The method of claim 1, wherein vital signs data for training the time series deep learning models is encoded using one or more of Min-Max scaling, lagged features, statistical features, a Fourier transform of input vital signs, to encode information in the frequency domain, one-hot encoding, and ordinal encoding.

6. The method of claim 5, further comprising presenting the output of the time series deep learning models in an integrated early warning dashboard.

7. The method of claim 6, further comprising providing a reinforcement learning agent which can use this human feedback on the recommended diagnostic and treatment plans to improve the future response of the Generative AI model.

8. The method of claim 1, wherein the time series deep learning models comprise one or more time series foundation models, and the method further comprises using Parameter-Efficient Fine-Tuning techniques to fine tune selected trainable parameters of these models for healthcare applications.

9. The method of claim 8, further comprising training a time series foundation model utilizing one or more Parameter-Efficient Fine-Tuning techniques comprising LoRA, VeRA, FourierFT.

10. The method of claim 2, wherein the vital signs forecasting head is adapted to forecast the patient vital signs into the future to predict sepsis or septic shock for the new

11. A system for early prediction of sepsis or septic shock through bio-data analysis on a computing device, the system adapted to:
train one or more time series deep learning models by:
encoding sepsis or septic shock patient vital signs as time series-based bio-data to generate embedded time series patches;
encoding associated patient medical histories utilizing a pre-trained language encoder to generate encoded patient medical history patches;
combining the embedded time series patches and the patient medical history patches into an array; and
randomly masking the embedded time series patches and asking the model to reconstructing the masked patches based at least in part on the associated patient medical histories; and
utilize the one or more trained time series deep learning models to predict sepsis or septic shock for a new patient.

12. The system of claim 11, wherein the system is further adapted to:
conduct a supervised training routine for one or more task heads layered on top of the one or more trained time series deep learning models, the one or more task heads comprising a vital signs forecasting head, a sepsis classification head, a shock anomaly detection head, and a report generation head.

13. The system of claim 12, wherein the supervised training routine comprises utilizing masked patches to make predictions about the patient's vital signs, and utilizing a learnable classification token to classify sepsis or septic shock.

14. The system of claim 13, wherein the supervised training for the report generation head comprises utilizing feature space mapping to generate a report comprising the predictions about the patient's vital signs, whether the patient is classified as having sepsis or septic shock, a summary of the patient's current medical condition, and recommendations for treatment and an action plan.

15. The system of claim 11, wherein vital signs data for training the time series deep learning models is encoded using one or more of Min-Max scaling, lagged features, statistical features, a Fourier transform of input vital signs, to encode information in the frequency domain, one-hot encoding, and ordinal encoding.

16. The system of claim 15, further comprising presenting the output of the time series deep learning models in an integrated early warning dashboard.

17. The system of claim 16, further comprising providing a reinforcement learning agent which can use this human feedback on the recommended diagnostic and treatment plans to improve the future response of the Generative AI model.

18. The system of claim 11, wherein the time series deep learning models comprise one or more time series foundation models, and the method further comprises using Parameter-Efficient Fine-Tuning techniques to fine tune selected trainable parameters of these models for healthcare applications.

19. The system of claim 18, further comprising training a time series foundation model utilizing one or more Parameter-Efficient Fine-Tuning techniques comprising LoRA, VeRA, FourierFT.

20. The system of claim 12, wherein the vital signs forecasting head is adapted to forecast the patient vital signs into the future to predict sepsis or septic shock for the new
